(19) United States
(12) Patent Application Publication
(10) Pub. No.: US 2021/0330844 A1
(43) Pub. Date: Oct. 28, 2021

(54) FIELD DEPLOYABLE MODULAR DECONTAMINATION STATION

(71) Applicant: MSI Defense Solutions, LLC, Mooresville, NC (US)

(72) Inventors: Maryn Merson HOLDEN, Davidson, NC (US); William Donald FERGUSON, JR., Charlotte, NC (US); David John HOLDEN, Davidson, NC (US)

(21) Appl. No.: 17/243,457

(22) Filed: Apr. 28, 2021

Related U.S. Application Data
(60) Provisional application No. 63/016,823, filed on Apr. 28, 2020.

(51) Int. Cl. A61L 2/22; A61L 2/24; A61L 2/26; E04H 1/12; B05B 16/80; B05B 16/40

(52) U.S. Cl. CPC A61L 2/22; A61L 2/24; A61L 2/26; E04H 1/1277; B05B 16/80; B05B 16/40; A61L 2202/26; A61L 2202/123; A61L 2202/14; A61L 2202/15; A61L 2202/16; A61L 2202/23; A61L 2202/24; A61L 2202/122

(57) ABSTRACT

A field deployable modular decontamination station includes a frame with a plurality of sprayers. The frame includes side panels and a top panel. The side panels and the top panel are configured to be attached together to create the frame. The side panels and the top panel are configured to be folded or disassembled for transportation or shipping. The plurality of sprayers are attached on an inside of the frame. The plurality of sprayers is configured to decontaminate a person or an object inside of the frame.

### CROSS-REFERENCE To RELATED APPLICATIONS

This application claims benefit to U.S. Provisional Patent Application No. 63/016,823, filed on Apr. 28, 2020, entitled “Field Deployable Modular Decontamination Station”, which is incorporated by reference in its entirety.

### FIELD OF THE DISCLOSURE

The present disclosure is directed to a means, system and method for decontamination, like decontamination of people, their clothes and accessories, and objects like baggage, shopping carts, the like, etc. More specifically, the present disclosure is directed to a field deployable modular decontamination station.

### BACKGROUND

In general, decontamination is known as the neutralization or removal of dangerous substances, radioactivity, or germs from an area, object, or person. Currently, in the early part of 2020, with the onset of the COVID-19 pandemic, without adequate testing or defense against a new strain of virus that to most is an “invisible enemy”, the public has been subject to stay at home orders in order to stem the spread of the virus and subsequent symptoms and illness. As such, there is clearly a need for more robust and adequate means for decontamination of the public.

Without any good high-volume, low-cost options to decontaminate ones' person or goods, citizens are urged or required to stay home, maintain a safe social distance, to wear masks, to subject themselves to temperature taking and contact tracing and so much more. Accordingly, the instant disclosure recognized that COVID-19 has brought to light a gap in the public's ability to have safe and decontaminated fellow shoppers, employees of the stores they shop, shopping carts, luggage, and other products, the like etc. Therefore, the disclosed field deployable modular decontamination station may be designed to be deployed to mitigate the effects of common influenzas, MERS, SARS, and H1N1 (Swine Flu) in addition to the most recent coronavirus, COVID-19 pandemic.

As a result, the instant disclosure may be designed to address at least certain aspects of the problems or needs discussed above by providing of the disclosed field deployable modular decontamination station or stations.

### SUMMARY

The present disclosure may solve the aforementioned limitations of the currently available decontamination devices, systems and/or methods by providing a field deployable modular decontamination station and a method of decontaminating a person or an object therewith.

Accordingly, in one aspect, the present disclosure embraces a field deployable modular decontamination station. The field deployable modular decontamination station may include a frame with a plurality of sprayers. The frame may include side panels and a top panel. The side panels and the top panel may be configured to be attached together to create the frame. The side panels and the top panel may be configured to be folded or disassembled for transportation or shipping. The plurality of sprayers may be attached on an inside of the frame. The plurality of sprayers may be configured to decontaminate a person or an object inside of the frame.

One feature of the disclosed field deployable modular decontamination station may be that each of the side panels and the top panel can include a transparent window. The transparent windows may be configured for observing the person or the object during the decontamination process on the inside of the frame.

Another feature of the disclosed field deployable modular decontamination station may be that the frame can further include a pan. The pan may be on the bottom of the frame. The pan may be configured to hold and support the frame in an unfolded or assembled configuration and in a folded or disassembled configuration.

Another feature of the disclosed field deployable modular decontamination station may be that the frame, including the side panels, the top panel and the pan, may be configured to be stored, transported, and deployed on a standard pallet.

Another feature of the disclosed field deployable modular decontamination station may be that the frame, including the side panels, the top panel and the pan, may be configured to be folded or disassembled by a single person, and/or unfolded or assembled by a single person.

Another feature of the disclosed field deployable modular decontamination station may be that the frame can be configured to be modular. In select embodiments, wherein the frame being modular may be configured to allow the frame to be connected to an adjacent frame in a side-by-side or parallel configuration. In other select embodiments, wherein the frame being modular may be configured to allow the frame to be connected in a front-to-back or series configuration. In select possibly preferred embodiments, wherein the frame being modular may be configured to allow the frame to be connected to an adjacent frame in the side-by-side or parallel configuration, and also connected in the front-to-back or series configuration.

In select embodiments of the disclosed field deployable modular decontamination station, each side panel can include a lower side panel and an upper side panel. In this configuration, the lower side panel and the upper side panel may be configured to be stacked on top of each other. Whereby, the frame is configured as a tall frame configuration. The tall frame configuration may be, but is clearly not limited thereto, a human configuration configured and sized for decontaminating humans.

In other select embodiments of the disclosed field deployable modular decontamination station, each side panel can include a single high side panel. Whereby, the frame is configured as a short frame configuration. The short frame configuration may be, but is clearly not limited thereto, a cart/gurney type configuration configured and sized for decontaminating shopping carts or hospital gurneys. In select embodiments, the cart/gurney type configuration of the frame that is configured and sized for decontaminating the shopping carts or the hospital gurneys can include a removable top portion. The removable top portion may be sized and configured for decontaminating the shopping carts or the hospital gurneys.

Another feature of the disclosed field deployable modular decontamination station may be that when the tall frame configuration is provided and each side panel includes the lower side panel and the upper side panel, the lower side panel or the upper side panel can be configured to be removable to create the short frame configuration with the single high side panel.

Another feature of the disclosed field deployable modular decontamination station may be that the tall frame configuration can be configured to be attached in a side-by-side or parallel configuration with adjacent frame configured as the short frame configuration.

In select embodiments of the disclosed field deployable modular decontamination station, the plurality of sprayers can include at least one top sprayer and at least one side sprayer. The at least one top sprayer may be attached to the top panel. The at least one top sprayer attached to the top panel may include a front elongated top spray bar and a back elongated spray bar. The front elongated top spray bar may be attached along a front top member on an inside of the top panel. The back elongated top spray bar may be attached along a back top member on the inside of the top panel. The at least one side sprayer may be attached to each of the side panels. The at least one side sprayer attached to each of the side panels may include a front elongated side spray bar and a back elongated side spray bar. The front elongated side spray bar may be attached along a front side member on an inside of the side panel. The back elongated side spray bar may be attached along a back side member on the inside of the side panel. In other select embodiments, the plurality of sprayers may further include at least one bottom sprayer. The at least one bottom sprayer may be positioned beneath a grate on the top of a pan at a bottom of the frame.

Another feature of the disclosed field deployable modular decontamination station may be the inclusion of a pump. The pump may be configured to spray a disinfectant/sanitizing agent out of the plurality of sprayers. In select embodiments, the pump may be attached to the frame at a bottom corner of one of the side panels.

Another feature of the disclosed field deployable modular decontamination station may be the inclusion of a card reader. The card reader may be configured to scan or read a card of the person or the object.

Another feature of the disclosed field deployable modular decontamination station may be the inclusion of a thermal scanning camera. The thermal scanning camera may be at a top configured to thermally scan the person or the object on the inside of the frame.

Another feature of the disclosed field deployable modular decontamination station may be the inclusion of enclosures on each side of the frame. The enclosures on each side of the frame may be configured to house the pump, and/or tanks configured to hold a disinfectant or a disinfectant/sanitizing agent.

Another feature of the disclosed field deployable modular decontamination station may be the inclusion of a foot pressure pad. The foot pressure pad may be at a bottom of the frame. The foot pressure pad may be configured to sense the presence of the person or the object on the inside of the frame.

Another feature of the disclosed field deployable modular decontamination station may be the inclusion of ramps. The ramps may be configured to provide a smooth surface from the ground to a pan at a bottom of the frame. The ramps may be designed and configured to prevent the person from tripping on the pan while entering the frame. The ramps may be designed and configured to let the object roll into the frame onto the pan from the ground.

In another aspect, the instant disclosure embraces the disclosed field deployable modular decontamination station in any of the embodiments and/or combination of embodiments shown and/or described herein.

In another aspect, the instant disclosure embraces a method of decontaminating a person or an object. The disclosed method of decontaminating a person or an object may generally include utilizing the disclosed field deployable modular decontamination station in any of the embodiments and/or combination of embodiments shown and/or described herein. As such, the disclosed method of decontaminating a person or an object may include the step of providing the disclosed field deployable modular decontamination station in any of the embodiments and/or combination of embodiments shown and/or described herein. With the provided field deployable modular decontamination, the disclosed method of decontaminating a person or an object may further include the steps of: transporting the disassembled field deployable modular decontamination station to a desired location; assembling the field deployable modular decontamination station at the desired location; positioning the person or the object on the inside the frame, and spraying the person or the object on the inside of the fame with the plurality of sprayers.

The foregoing illustrative summary, as well as other exemplary objectives and/or advantages of the disclosure, and the manner in which the same are accomplished, are further explained within the following detailed description and its accompanying drawings.

### BRIEF DESCRIPTION OF THE DRAWINGS

The present apparatuses, systems and methods will be better understood by reading the Detailed Description with reference to the accompanying drawings, which are not necessarily drawn to scale, and in which like reference numerals denote similar structure and refer to like elements throughout, and in which:

FIG. 1 is a front perspective view of the disclosed field deployable modular decontamination station according to select embodiments of the instant disclosure with a person or human inside the frame in an unfolded or assembled configuration and a tall or human configuration;

FIG. 2 is a side view of the field deployable modular decontamination station of FIG. 1 with the person or human inside the frame;

FIG. 3 is a front view of the field deployable modular decontamination station of FIG. 1 with the person or human inside the frame;

FIG. 4 is a top view of the field deployable modular decontamination station of FIG. 1 with the person or human inside the frame;

FIG. 5 shows another front perspective view of the field deployable modular decontamination station of FIG. 1 in use with the person or human inside the frame being sprayed with a disinfectant/sanitizing agent;

FIG. 6 shows a front perspective view of the field deployable modular decontamination station of FIG. 1 where it is shown on a pallet in a folded or disassembled configuration;

FIG. 7 shows a side view of the field deployable modular decontamination station of FIG. 6 where it is shown on a pallet in a folded or disassembled configuration;

FIG. 8 shows a perspective partially disassembled view of the disclosed field deployable modular decontamination station of FIG. 1 with a person or human positioned inside of the frame;

FIG. 9 shows a side perspective view of one of the side panels or top panels of the frame of the disclosed field deployable modular decontamination station according to select embodiments of the instant disclosure;

FIG. 10 shows a perspective view of two of the disclosed field deployable modular decontamination station units of FIG. 1 set up in series with persons or humans walking through each unit;

FIG. 11 shows a perspective view of two of the disclosed field deployable modular decontamination station units of FIG. 1 set up in parallel with persons or humans walking through each unit;

FIG. 12 is a zoomed in front view of the disclosed field deployable modular decontamination station according to select embodiments of the instant disclosure showing a possible pump and location of the pump;

FIG. 13 is a zoomed in perspective side view of the disclosed field deployable modular decontamination station unit of FIG. 12 showing the possible pump and location of the pump according to select embodiments of the instant disclosure;

FIG. 14 is a front perspective view of the disclosed field deployable modular decontamination station unit according to select embodiments of the instant disclosure in a short or cart/gurney configuration with a shopping cart being positioned therein the frame;

FIG. 15 is a side perspective view of the disclosed field deployable modular decontamination station unit according to select embodiments of the instant disclosure in a short or cart/gurney configuration with a shopping cart being positioned therein the frame and an enclosure on one side of the frame for housing the pump and/or tanks for holding the disinfectant/sanitizing agent;

FIG. 16 is a front view of the disclosed field deployable modular decontamination station unit of FIG. 14 in the short or cart/gurney configuration with the shopping cart being positioned therein the frame;

FIG. 17 is a top view of the disclosed field deployable modular decontamination station unit of FIG. 15 in the short or cart/gurney configuration with the shopping cart being positioned therein the frame;

FIG. 18 is a side view of the disclosed field deployable modular decontamination station unit of FIG. 15 in the short or cart/gurney configuration with the shopping cart being positioned therein the frame;

FIG. 19 is a side perspective view of 2 of the disclosed field deployable modular decontamination station units set up in parallel orientation according to select embodiments of the instant disclosure, with one of the field deployable modular decontamination station units being set up in the tall or human configuration (like as shown in FIGS. 1-8) and an adjacent field deployable modular decontamination station unit being set up in a short or cart/gurney configuration (like as shown in FIGS. 14-18);

FIG. 20 is a side perspective view of the disclosed field deployable modular decontamination station unit according to select embodiments of the instant disclosure in a short or cart/gurney configuration with a hospital gurney being positioned therein the frame;

FIG. 21 is a front view of the field deployable modular decontamination station unit of FIG. 20 in the short or cart/gurney configuration with the hospital gurney being positioned therein the frame;

FIG. 22 is a side view of the field deployable modular decontamination station unit of FIG. 20 in the short or cart/gurney configuration with the hospital gurney being positioned therein the frame;

FIG. 23 shows a front perspective view of the disclosed field deployable modular decontamination station according to select embodiments of the instant disclosure with a card reader and ramps in use with the person or human inside the frame being sprayed with a disinfectant/sanitizing agent;

FIG. 24 shows a side perspective view of the disclosed field deployable modular decontamination station according to select embodiments of the instant disclosure with a foot pressure pad, card reader, side enclosure, and thermal scanning camera on the top with the person or human inside the frame;

FIG. 25 shows a front perspective view of the disclosed field deployable modular decontamination station according to select embodiments of the instant disclosure with a card reader, ramps, and enclosure on each side in use with the person or human inside the frame being sprayed with a disinfectant/sanitizing agent; and

FIG. 26 is a flow chart diagram of the disclosed method of decontaminating a person or an object according to select embodiments of the instant disclosure.

It is to be noted that the drawings presented are intended solely for the purpose of illustration and that they are, therefore, neither desired nor intended to limit the disclosure to any or all of the exact details of construction shown, except insofar as they may be deemed essential to the claimed disclosure.

### DETAILED DESCRIPTION

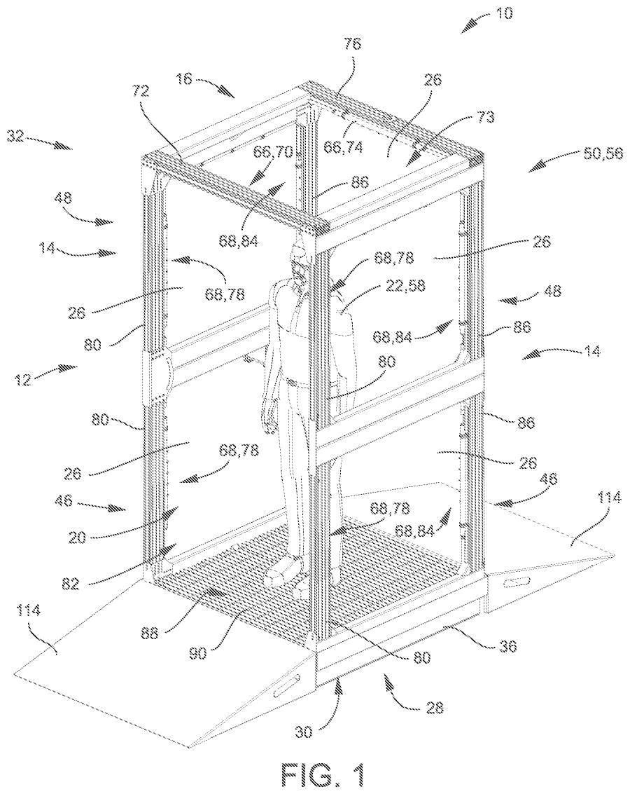

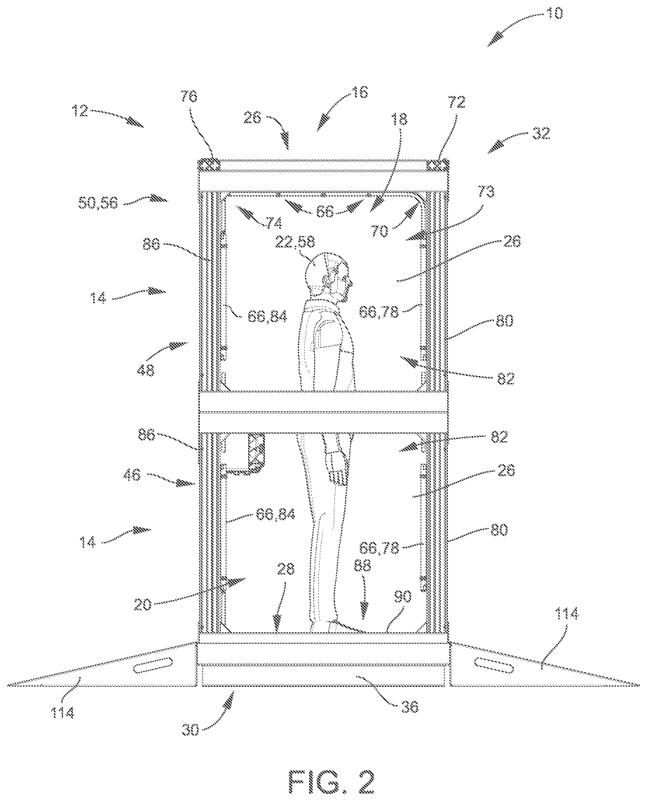

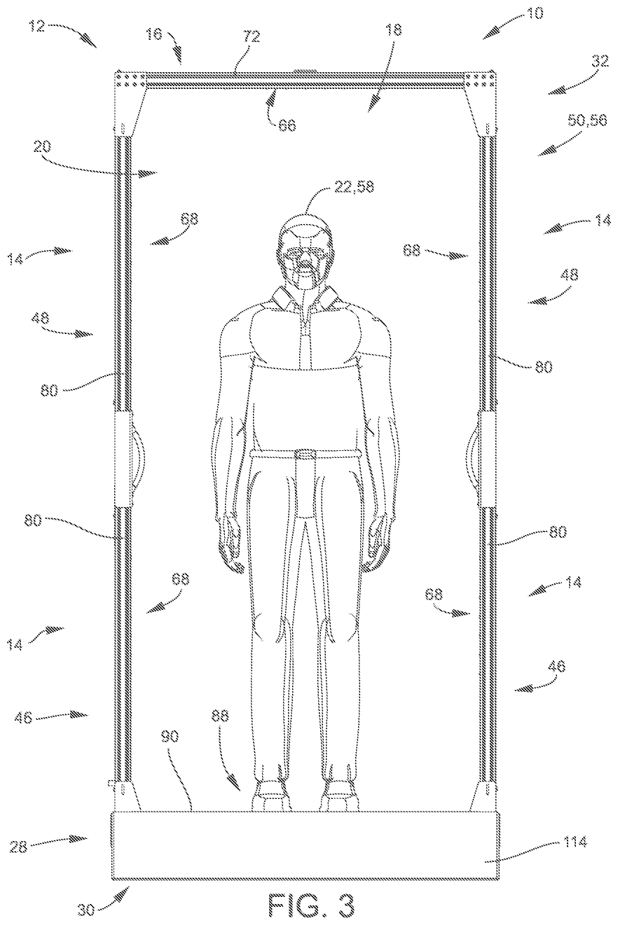

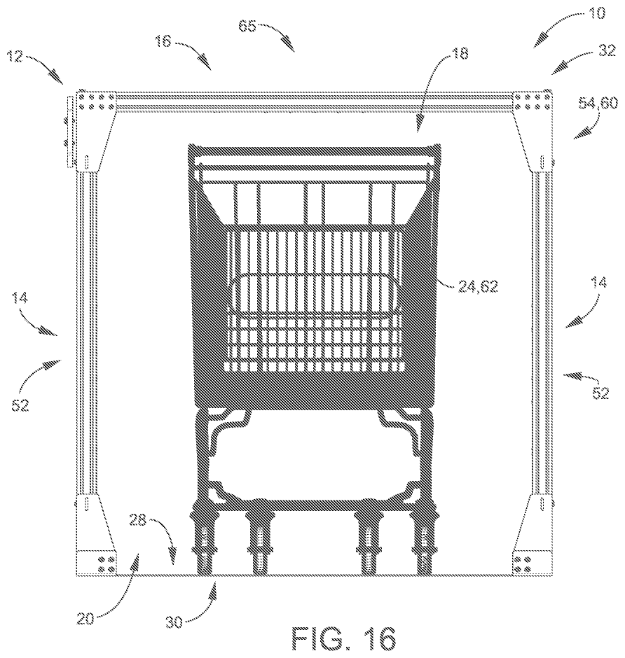

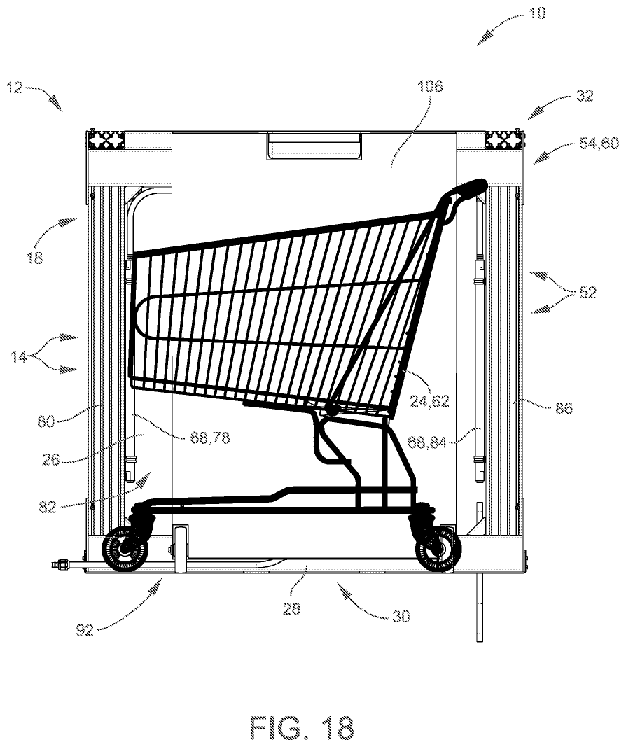

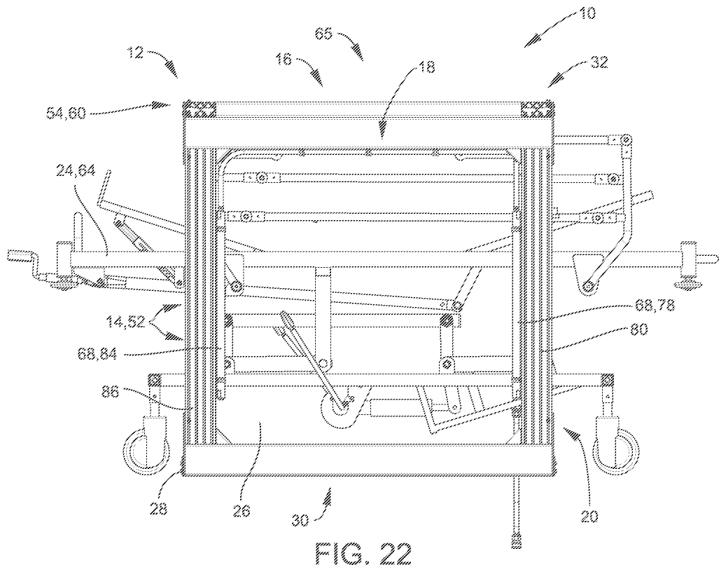

Referring now to FIGS. 1-26, in describing the exemplary embodiments of the present disclosure, specific terminology is employed for the sake of clarity. The present disclosure, however, is not intended to be limited to the specific terminology so selected, and it is to be understood that each specific element includes all technical equivalents that operate in a similar manner to accomplish similar functions. Embodiments of the claims may, however, be embodied in many different forms and should not be construed to be limited to the embodiments set forth herein. The examples set forth herein are non-limiting examples and are merely examples among other possible examples.

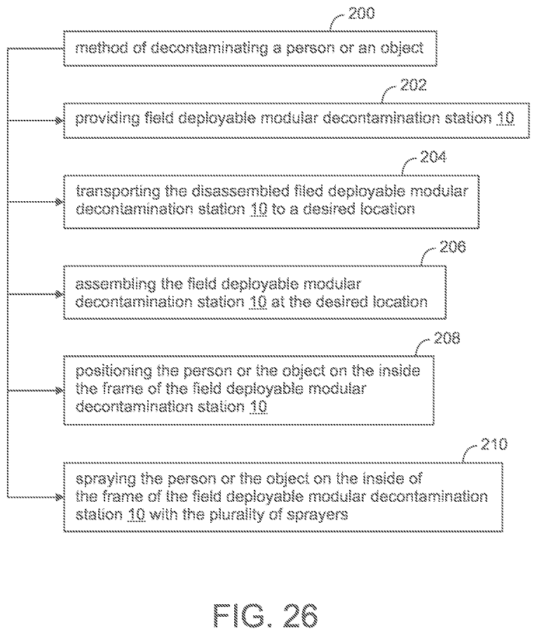

Referring now specifically to FIGS. 1-25, the instant disclosure may address at least certain aspects of the above-mentioned needs, issues and/or problems and may provide field deployable modular decontamination station 10. The present disclosure of field deployable modular decontamination station 10 may solve the aforementioned limitations of the currently available decontamination devices, systems and/or methods by providing field deployable modular decontamination station 10 (see FIGS. 1-25) and method 200 of decontaminating person 22 and/or object 24 therewith (see FIG. 26).

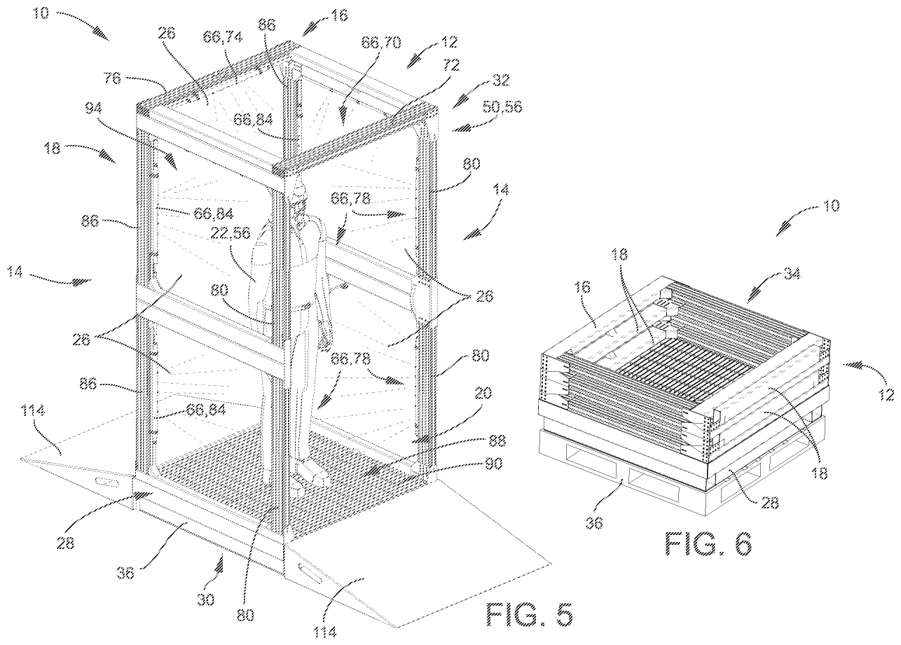

Field deployable modular decontamination station 10 may generally include frame 12 with plurality of sprayers 18, like a plurality of spraying nozzles. Frame 12 may provide the structure of field deployable modular decontamination station 10 for positioning sprayers 18 around person 22 and/or object 24 within frame 12. Frame 12 may include any parts and/or components for providing such structure of field deployable modular decontamination station 10 for positioning sprayers 18 around person 22 and/or object 24 within frame 12 As shown in the Figures, frame 12 may include side panels 14 and top panel 16. Side panels 14 and top panel 16 may be configured to be attached together to create frame 12. Side panels 14 and top panel 16 may be configured to be folded or disassembled for transportation or shipping. The plurality of sprayers 18 may be attached on inside 20 of frame 12. The plurality of sprayers 18 may be configured to decontaminate person 22 and/or object 24 inside of frame 12, like by spraying disinfectant/sanitizing agent 94 on such person 22 and/or object 24 inside of frame 12 (see FIGS. 5, 15, 23 and 25).

One feature of field deployable modular decontamination station 10 may be that each of the side panels 14 and the top panel 16 can include transparent window 26. Transparent window 26 may be fully transparent (to fully see the person 22 and/or object 24 inside frame 12) and/or partially transparent (like, to give the person 22 privacy within field deployable modular decontamination station 10). As such, transparent windows 26 may be configured for observing person 22 and/or object 24 during the decontamination process on inside 20 of frame 12.

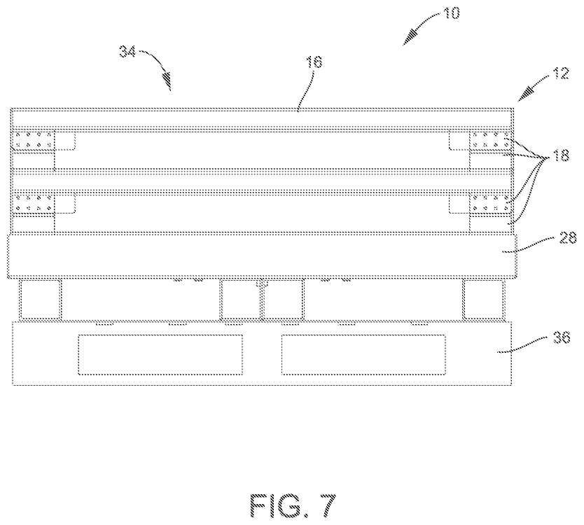

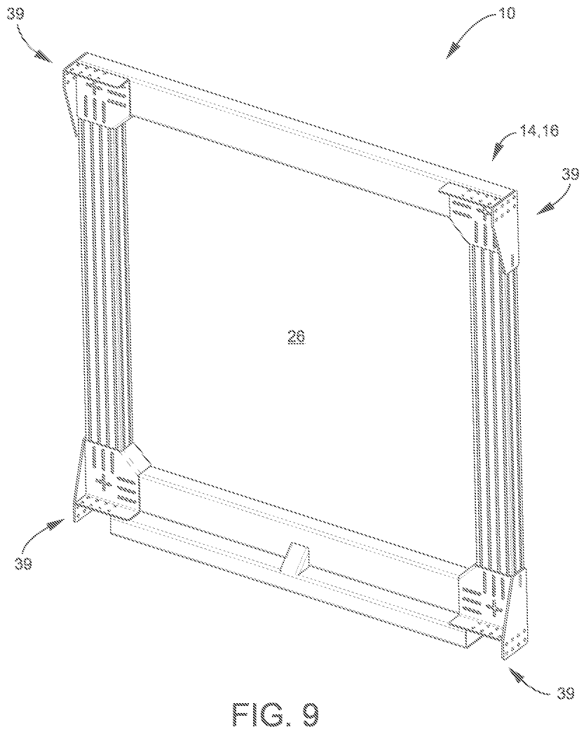

Another feature of field deployable modular decontamination station 10 may be that frame 12 can further include pan 28. Pan 28 may be on bottom 30 of frame 12. Pan 28 may be configured to hold and support frame 12 in unfolded or assembled configuration 32 (see FIGS. 1-5, 8, and 10-25) and in folded or disassembled configuration 34 (see FIGS. 6-7). Pan 28 may be sized and configured to provide the base or support for frame 12, including side panels 14 and top panel 16. Together or in combination with top panel 16, pan 28 may provide the support for erecting side panels 14 of frame 12. As such, bottom pan 28 and top panel 16 may be configured as stabilizing panels to side panels 14. In addition, as shown in FIGS. 6-7, pan 28 may be sized and configured to where side panels 14 and top panel 16 fit as they are disassembled (unattached or unfolded), like for storage and/or transportation on standard pallet 36. As a result, another feature of field deployable modular decontamination station 10 may be that frame 12, including, but not limited to, side panels 14, top panel 16 and pan 28, may be configured to be stored, transported, and deployed on standard pallet 36. Another feature of field deployable modular decontamination station 10 may be that frame 12, including, but not limited to, side panels 14, top panel 16 and pan 28, may be configured to be folded or disassembled by a single person, and/or unfolded or assembled by a single person. Referring to FIG. 9, side panel 14 and/or top panel 16 is shown. As such, in select embodiments side panels 14 and top panels 16 may be similar. As should be clearly understood by one in the art, a single person may lift and secure each of these panels in position on frame 12. As shown in FIG. 9, a plurality of brackets 39 may be included, like on each corner of the panels, for securing adjacent panels to one another or for securing the panels to pan 28. Any type of fasteners (screws, bolts, the like, etc.) may be utilized for securing adjacent panels via brackets 39.

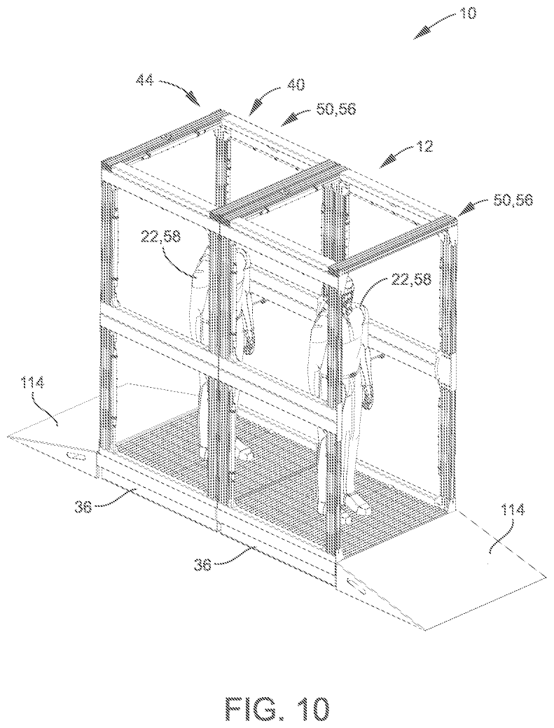

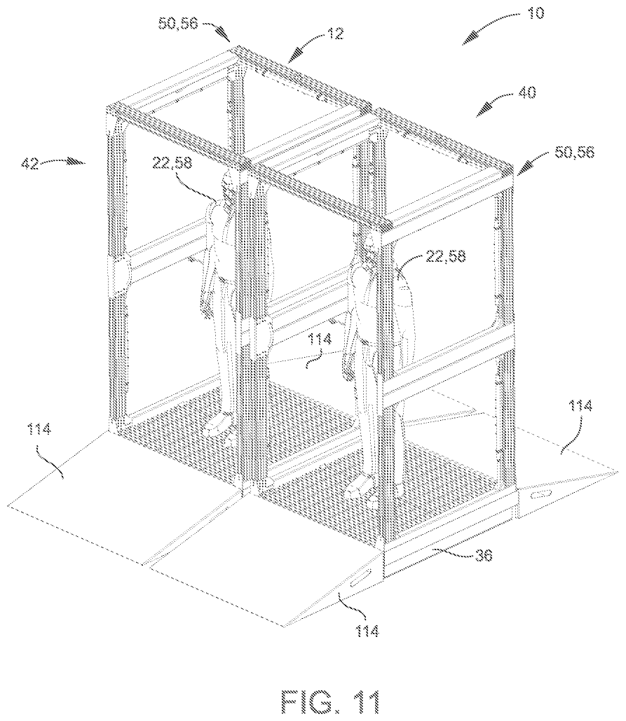

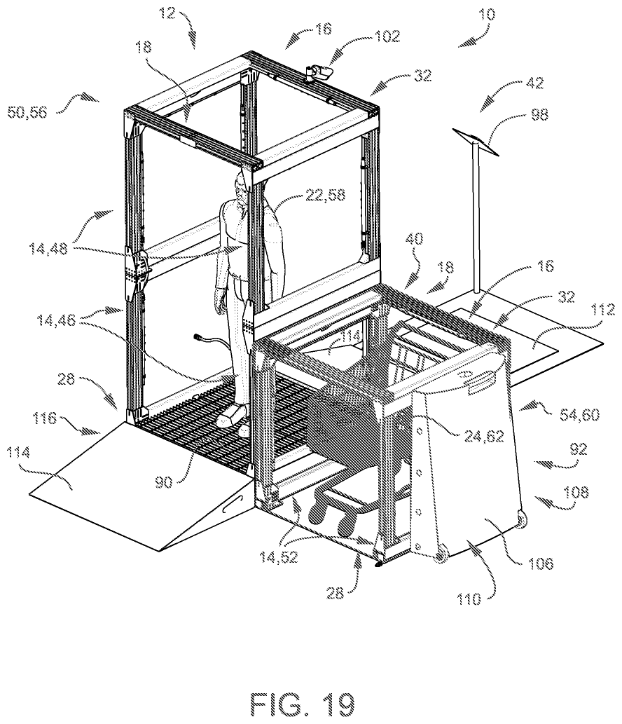

Referring specifically to FIGS. 10, 11 and 19, another feature of field deployable modular decontamination station 10 may be that frame 12 can be configured to be modular. Frame 12 being modular may be designed to mean that frame 12 can be connected to other or adjacent frames 40 in various configurations or setups. In select embodiments, frame 12 being modular may be configured to allow frame 12 to be connected to at least one adjacent frame 40 in side-by-side or parallel configuration 42 (see FIGS. 11 and 19). In other select embodiments, frame 12 being modular may be configured to allow frame 12 to be connected in front-to-back or series configuration 44 (see FIG. 10). In select possibly preferred embodiments, frame 12 being modular may be configured to allow frame 12 to be connected to adjacent frame 40 in side-by-side or parallel configuration 42, and also connected in front-to-back or series configuration 44. As such, the user or owner of field deployable modular decontamination station 10 may have options on the style or setup of using multiple frames in their setup of field deployable modular decontamination station 10.

Referring now specifically to FIGS. 1-5, in select embodiments of field deployable modular decontamination station 10, each side panel 14 can include lower side panel 46 and upper side panel 48. In this configuration, lower side panel 46 and upper side panel 48 may be configured to be stacked on top of each other. Whereby, frame 12 can be configured as tall frame configuration 50. Tall frame configuration 50 may be, but is clearly not limited thereto, human configuration 56 configured and sized for decontaminating humans 58, as shown in the Figures.

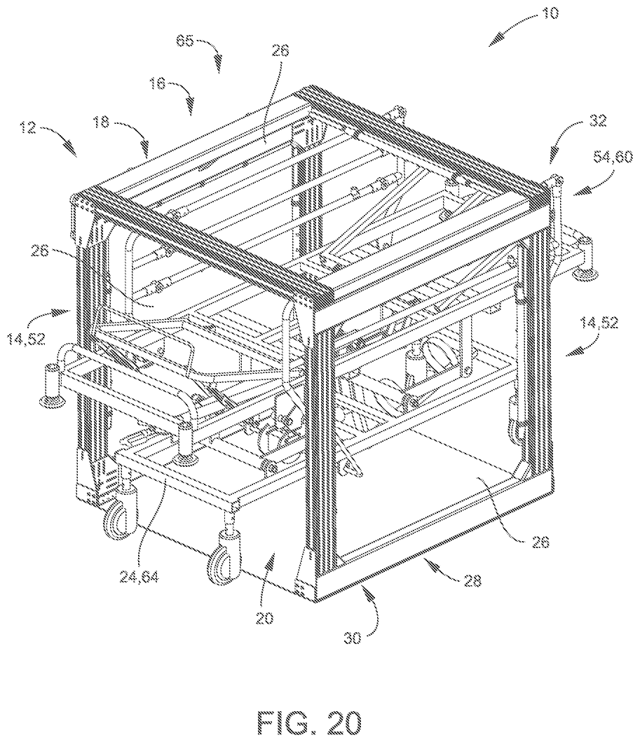

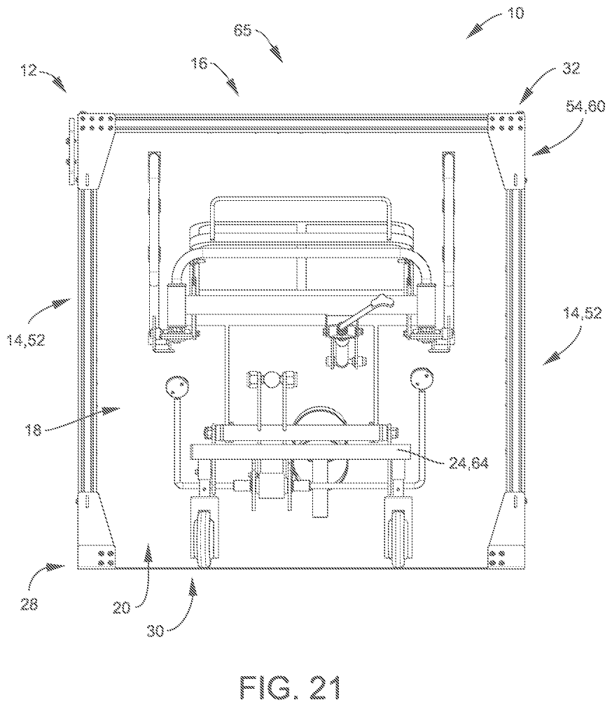

Referring now specifically to FIGS. 14-22, in other select embodiments of field deployable modular decontamination station 10, each side panel 14 can include single high side panel 52. Whereby, frame 12 can be configured as short frame configuration 54. Short frame configuration 54 may be, but is clearly not limited thereto, cart/gurney type configuration configured 60, as shown the Figures, which can be sized for decontaminating shopping carts 62 (see FIGS. 14-19) or hospital gurneys 64 (see FIGS. 20-22). In select embodiments, cart/gurney type configuration 60 of frame 12 that is configured and sized for decontaminating shopping carts 62 and/or hospital gurneys 64 can include removable top portion 65. Removable top portion 65 may be sized and configured for accessing and decontaminating shopping carts 62 and/or hospital gurneys 64 while inside frame 12. As should be understood by the embodiments shown in the Figures, another feature of field deployable modular decontamination station 10 may be that when tall frame configuration 50 is provided and each side panel 14 includes lower side panel 46 and upper side panel 48, lower side panel 46 or upper side panel 48 can be configured to be removable to create short frame configuration 54 with just single high side panel 52. As such, the user or owner of field deployable modular decontamination station 10 may assemble frame 12 in the desired form of tall frame configuration 50 or short frame configuration 54. Referring specifically to FIG. 19, another feature of field deployable modular decontamination station 10, may be that, in select embodiments, tall frame configuration 50 can be configured to be attached in side-by-side or parallel configuration 42 with adjacent frame 40 configured as short frame configuration 54, or vice versa.

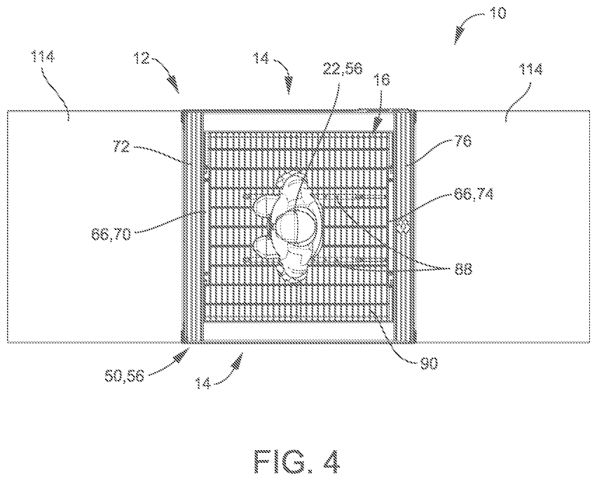

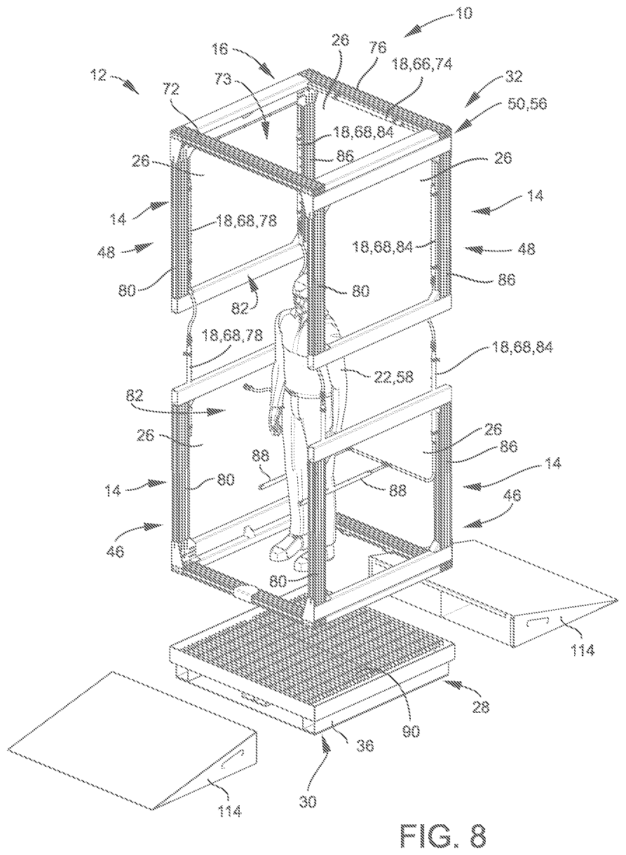

As shown in the Figures, field deployable modular decontamination station 10 may include plurality of sprayers 18. Sprayers 18 may be configured for spraying disinfectant/sanitizing agent 94, or the like, all over person 22 and/or object 24 inside of frame 12. The plurality of sprayers may include any number, size, configuration and/or location of sprayers 18 within or on frame 12. In select embodiments, the plurality of sprayers 18 can include at least one top sprayer 66 and at least one side sprayer 68. The at least one top sprayer 66 may be attached to top panel 16. In select embodiments, the at least one top sprayer 66 attached to top panel 16 may include front elongated top spray bar 70 and back elongated spray bar 74. Front elongated top spray bar 70 may be attached along front top member 72 on inside 73 of top panel 16. Back elongated top spray bar 74 may be attached along back top member 76 on inside 73 of top panel 16. The at least one side sprayer 68 may be attached to each of the side panels 14. In select embodiments, the at least one side sprayer 68 attached to each of the side panels 14 may include front elongated side spray bar 78 and back elongated side spray bar 84. The front elongated side spray bar 78 may be attached along front side member 80 on inside 82 of each side panel 14. Back elongated side spray bar 84 may be attached along back side member 86 on inside 82 of each side panel 14. As best shown in FIGS. 4 and 8, in other select embodiments, the plurality of sprayers 18 may optionally include at least one bottom sprayer 88. The at least one bottom sprayer 88 may be positioned beneath grate 90 on top of pan 28 at bottom 30 of frame 12.

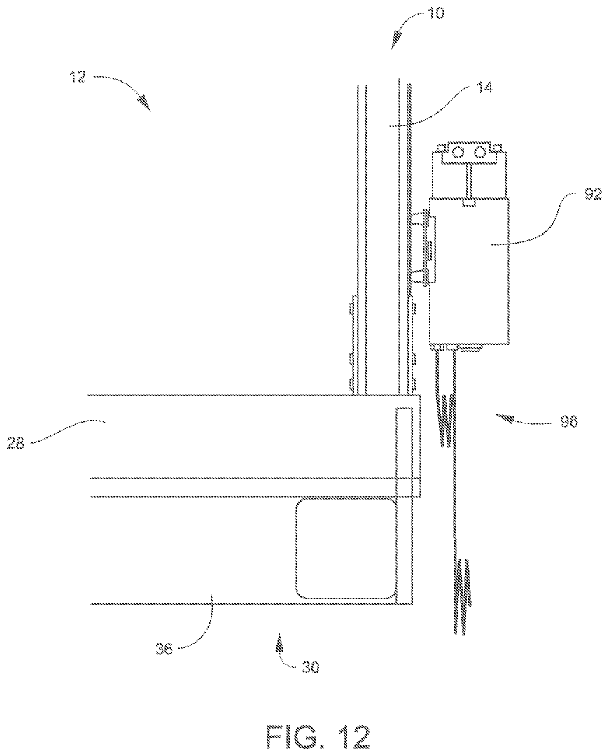

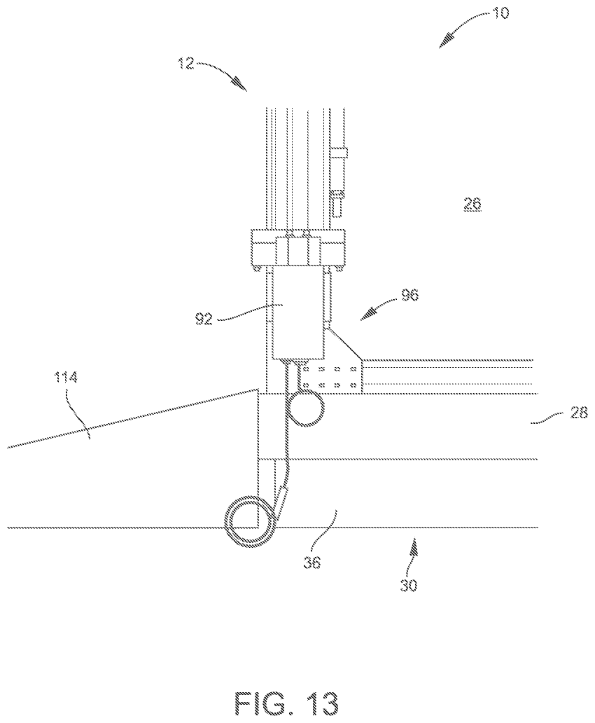

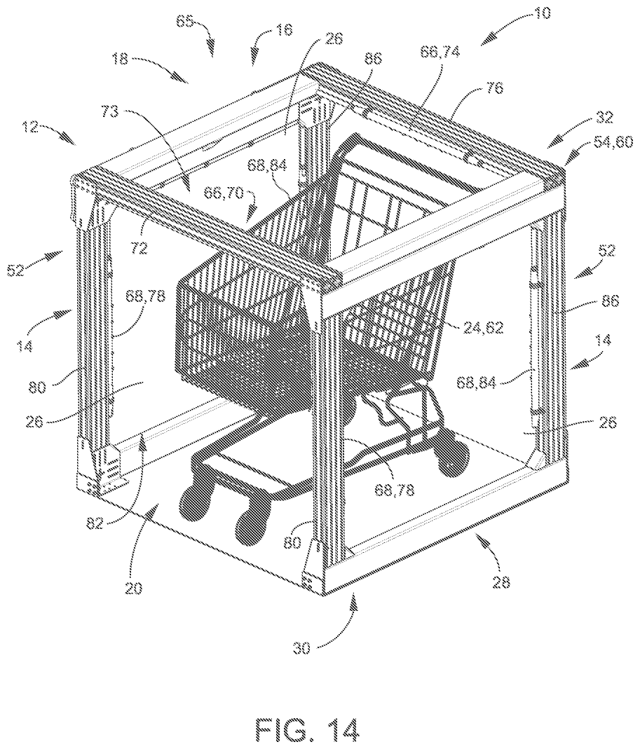

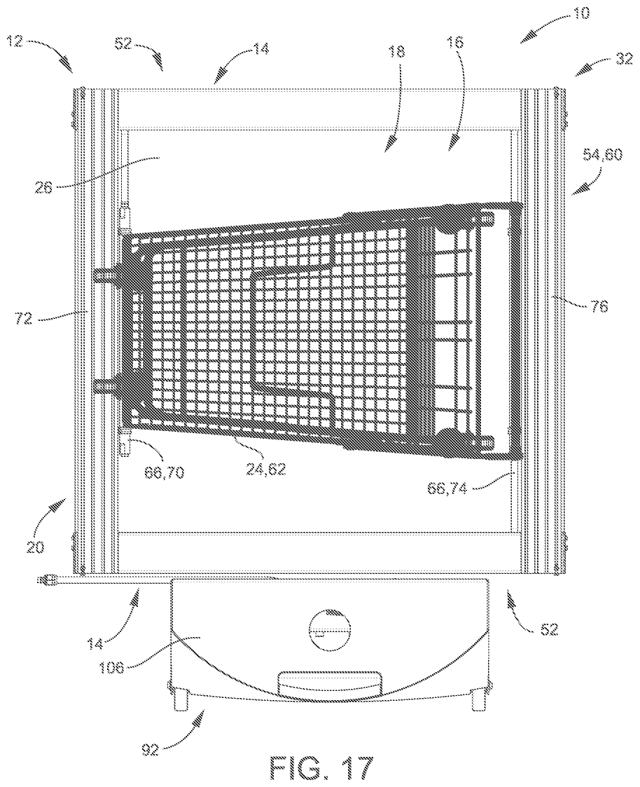

Referring now specifically to FIGS. 12, 13, 17-19, and 24-25, another feature of field deployable modular decontamination station 10 may be the inclusion of pump 92. Pump 92 may be configured to spray disinfectant/sanitizing agent 94, or the like, out of the plurality of sprayers 18. Pump 92 may include any conduits, tubes, connections, the like, etc., for moving disinfectant/sanitizing agent 94, or the like, from tanks 110 to the plurality of sprayers 18. Pump 92 may be sized and configured to provide the desired amount and pressure of disinfectant/sanitizing agent 94, or the like out of the plurality of sprayers 18. Referring now specifically to FIGS. 12 and 13, in select embodiments, pump 92 may be attached to frame 12 at, or approximate to bottom corner 96 of one of side panels 14. In other select embodiments, referring to FIGS. 17-19 and 24-25, pump 92 may be housed in enclosure 106 housed on one of sides 108 of frame 12.

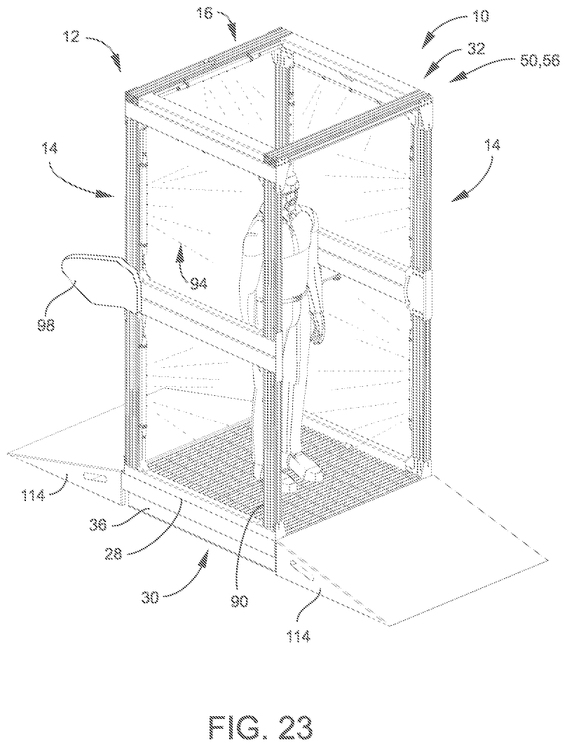

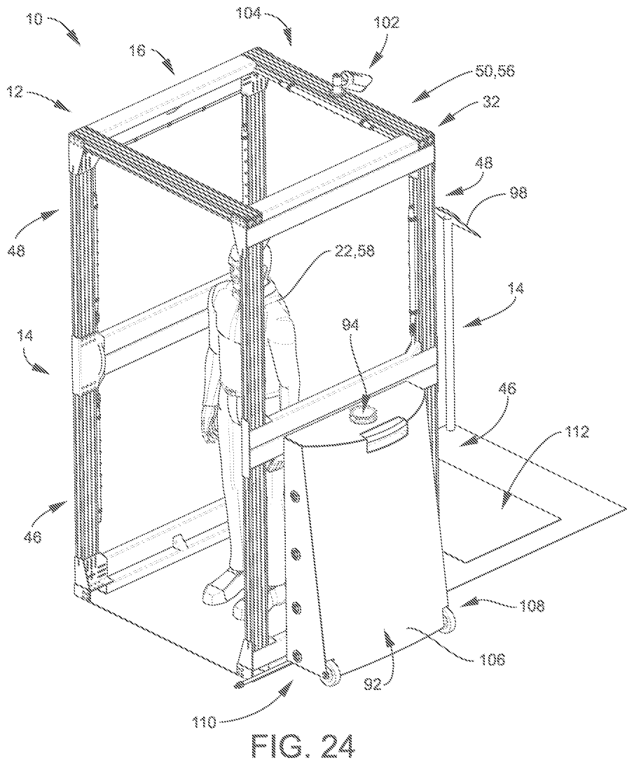

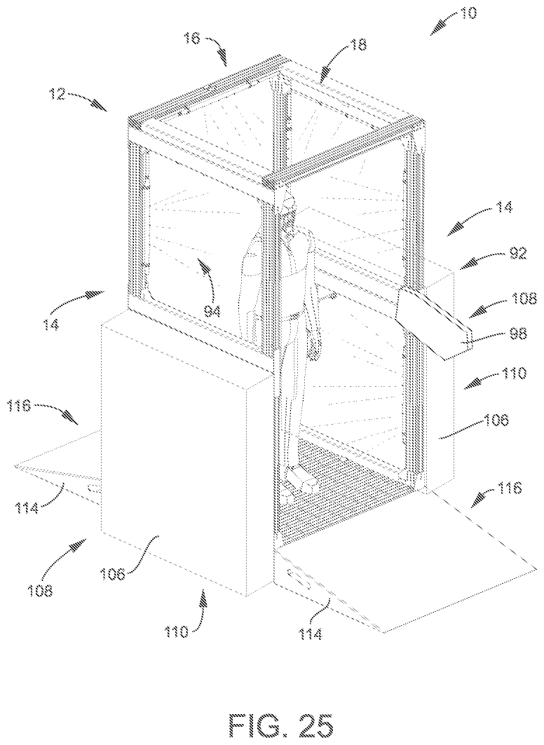

Referring now specifically to FIGS. 23-25, another feature of field deployable modular decontamination station 10 may be the inclusion of an optional card reader 98. Card reader 98 may be configured to scan or read a card, or the like, of the person 22 and/or the object 24, like for identification and/or tracking purposes. As shown in FIGS. 23 and 25, in select embodiments, card reader 98 may be attached to frame 12 of field deployable modular decontamination station 10, like on the entrance side of frame 12. As shown in FIG. 24, in select other embodiments, card reader 98 can be positioned adjacent frame 12, like with foot pressure pad 112 or ramp 114.

Referring now specifically to FIGS. 19 and 24, another feature of field deployable modular decontamination station 10 may be the inclusion of an optional thermal scanning camera 102. Thermal scanning camera 102 may be positioned anywhere on or around frame 12, including, in select possibly preferred embodiments, being positioned on top 104. Thermal scanning camera 102 may be configured to thermally scan person 22 and/or object 24 on inside 20 of frame 12, like for checking temperatures or the like.

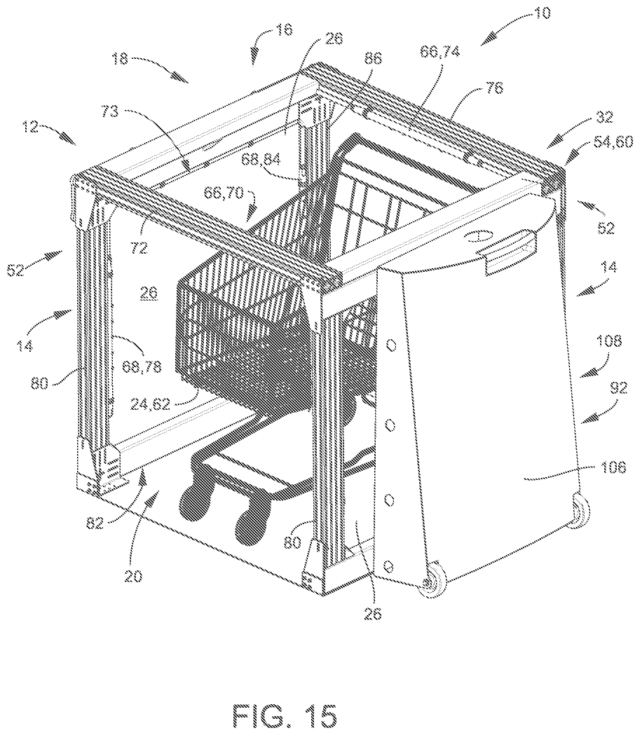

Referring now specifically to FIGS. 15, 17-19, and 24-25, another feature of field deployable modular decontamination station 10 may be the inclusion of optional enclosures 106 on one or each side 108 of frame 12. Enclosures 106 on one or each side 108 of frame 12 may be configured to house pump 92, and/or tanks 110 configured to hold disinfectant/sanitizing agent 94, or the like. Enclosure 106 may also be configured to house any control systems, computers, or processors configured to control and manage field deployable modular decontamination station 10.

Referring now specifically to FIGS. 19 and 24, another feature of field deployable modular decontamination station 10 may be the inclusion of optional foot pressure pad 112. Foot pressure pad 112 may be at bottom 30 of frame 12, or approximate thereto. Foot pressure pad 112 may be configured to sense the presence of person 22 and/or object 24 on inside 20 of frame 12.

Referring now specifically to FIGS. 1-5, 8, 10-11, 13, 19, 23 and 25, another feature of deployable modular decontamination station 10 may be the inclusion of ramps 114. Ramps 114 may be configured to provide smooth surface 116 from the ground to pan 28 at bottom 30 of frame 12. Ramps 114 may be designed and configured to prevent person 22 from tripping on pan 28 while entering frame 12. Ramps 114 may also be designed and configured to let object 24 roll into frame 12 onto pan 28 from the ground.

In another aspect, the instant disclosure embraces field deployable modular decontamination station 10 in any of the embodiments and/or combination of embodiments shown and/or described herein.

Referring now to FIG. 26, in another aspect, the instant disclosure embraces method 200 of decontaminating person 22 and/or object 24, the like, etc. Method 200 of decontaminating person 22 and/or object 24 may generally include utilizing the disclosed field deployable modular decontamination station 10 in any of the embodiments and/or combination of embodiments shown and/or described herein. As such, method 200 of decontaminating person 22 and/or object 24 may include step 202 of providing field deployable modular decontamination station 10 in any of the embodiments and/or combination of embodiments shown and/or described herein. With the provided field deployable modular decontamination 10, method 200 of decontaminating person 22 and/or object 24 may further include the steps of: step 204 of transporting the disassembled field deployable modular decontamination station 10 to a desired location (grocery store, shopping mall, hospital, office, residence, military base, the like, etc.); step 206 of assembling field deployable modular decontamination station 10 at the desired location; step 208 of positioning person 22 and/or object 24 on the inside of frame 12, and step 210 of spraying person 22 and/or object 24 on the inside of fame 12 with the plurality of sprayers 18, like with disinfectant/sanitizing agent 94.

In sum, as shown in the Figures, field deployable modular decontamination station 10 may be designed to provide a capacity for every and any business, any place of work, any place of social gathering, any operating enterprise or entity to apply decontamination product evenly and routinely to persons and their property (boxes, carts, personal belongings, etc.). Its design enables it to be configured in series (with similar field deployable modular decontamination station units, with other devices like metal detectors, with thermal cameras, with ID badge scanners, and the design enables multiple units to be configured to be high-capacity (multi-axial passage, side-by-side) or extended decontamination (end-to-end, single-axis passage with an extended decontamination runway) mode.

One advantage of field deployable modular decontamination station 10 may be that it can feature pan 28 into which the panels (side panels 14 and top panel 16) fit as they are assembled as shown in FIG. 1 and FIG. 8, showing the different stages of assembly.

Another advantage of field deployable modular decontamination station 10 may be that, as shown in FIG. 8, the bottom pan 28 and top panel 16 are stabilizing panels to the side panels 14 (which can be a single configuration used four (4) times). As examples, and clearly not limited thereto, these panels (side panels 14 and top panel 16) may be made, in one embodiment, out of extruded 80/20 brand slotted extruded aluminum with transparent window 26 therein.

Another advantage of field deployable modular decontamination station 10 may be that sprayers 18 which dispense disinfectant/sanitizing agent 94 may be attach to the extruded tracking tubing of frame 12 and can be re-positioned for optimum angle/height/pattern/personnel /package dimensions. As shown in FIGS. 5, 15, 23 and 25, elongated spray bars (70, 74, 78 and/or 84) are shown spraying person 22 or human 58 in frame 12.

Another advantage of field deployable modular decontamination station 10 may be that, when placed side-by-side or end-to-end the units can be attached together for stability and rigidity using simple commercially available fasteners and gusset plates.

Another advantage of field deployable modular decontamination station 10 may be that ramps 114 may be made to interface to the lips of pan 28 for stability and when configuring in end-to-end configuration ramps 114 may be simply removed from one end and placed on the other end.

Another advantage may be that field deployable modular decontamination station 10 may be independent and agnostic as to what decontamination/sanitizing agent 94 is used and can be configured to use of different agents and used at different rates because sprayers 18, like elongated spray bars (70, 74, 78 and/or 84), may have thread in spray nozzles using commercially available fittings. These may allow the users to adjust for the amount of material flowrate desired, if they so choose, and the spray coverage.

Another advantage of field deployable modular decontamination station 10 may be that transparent window 26 of side panels 14 of frame 12 can have either clear (lexan or similar) sides to enable people to see that person 22 or human 58 in frame 12 is following instructions or can be configured with solid sides to allow privacy. Branding or advertisements could be added on these sides as desired.

Another advantage of field deployable modular decontamination station 10 may be that, as best shown in FIGS. 5, 15, 23 and 25, there are spray bars (70, 74, 78 and/or 84), on the side, top and bottom underneath grate 90, which spray all over the user, including their upper body and head area, and lower leg area.

Another advantage of field deployable modular decontamination station 10 may be that, referring now to FIGS. 6 and 7, the modular nature of the panels which may break down and nest into the lower pan 28 may also enable the unit to pack down into a common shipping form, like on standard pallet 36 (for the personnel station, a common pallet size of 48″×48″ may be used). This enables multiple units to be stacked securely for shipping and may maximize shipping efficiency. Accordingly, FIG. 5 shows an image of the unit fully assembled and operational next to FIG. 6 showing the unit in its broken-down state ready for shipping. Unlike other systems that people may have been building out of wood or welded frames the ability to break down and ship or store away until next “pandemic season”, this mobility feature of field deployable modular decontamination station 10 can offer a tremendous economy of storage and shipping and logistics. Adding to that efficiency is short frame configuration 54 or cart/gurney configuration 60, like for shopping cart sizing in the top section.

Another advantage of field deployable modular decontamination station 10 may be that, referring to FIGS. 12-13, pump 92 is shown for pumping the disinfectant/sanitizing agent 94 through sprayers 18 on frame 12. Pump 92 and/or accompanying tanks 110 of disinfectant/sanitizing agent 94 can be attached to frame 12, like as shown in FIGS. 12-13 and/or FIGS. 15, 17-19, and 24-25, or they could be independent of the system and be placed next to the system or in a remote location.

Another advantage of field deployable modular decontamination station 10 may be that, referring now to FIGS. 14-22, aiding in the further modularity, the top section of field deployable modular decontamination station 10, or upper side panels 48, may be designed to incorporate the dimensions and features to decontaminate common shopping carts 62, hospital gurneys 64, and/or the like. This may be accomplished by removing lower side panels 46 from frame 12. Though they may vary in dimension, the spray bar nature and the use of spraying nozzles, enables a multitude of shopping cart size and configurations to be decontaminated.

Another advantage of field deployable modular decontamination station 10 may be that, referring now to FIGS. 19 and 24, in select embodiments, foot pressure pad 112 may be included. Foot pressure pad 112 may be at bottom 30 of the unit for sensing the presence of person 22 and/or object 24 to be decontaminated or sanitized.

Another advantage of field deployable modular decontamination station 10 may be that, referring now to FIGS. 23 and 25, in select embodiments, card reader 98 may be included for controlling field deployable modular decontamination station unit 10.

Another advantage of field deployable modular decontamination station 10 may be that, referring now to FIGS. 19 and 24, in select embodiments, thermal scanning camera 102 may be included, like at top 104 of the unit, for scanning the person prior to or during use of the field deployable modular decontamination station unit 10.

In the specification and/or figures, typical embodiments of the disclosure have been disclosed. The present disclosure is not limited to such exemplary embodiments. The use of the term “and/or” includes any and all combinations of one or more of the associated listed items. The figures are schematic representations and so are not necessarily drawn to scale. Unless otherwise noted, specific terms have been used in a generic and descriptive sense and not for purposes of limitation.

The foregoing description and drawings comprise illustrative embodiments. Having thus described exemplary embodiments, it should be noted by those skilled in the art that the within disclosures are exemplary only, and that various other alternatives, adaptations, and modifications may be made within the scope of the present disclosure. Merely listing or numbering the steps of a method in a certain order does not constitute any limitation on the order of the steps of that method. Many modifications and other embodiments will come to mind to one skilled in the art to which this disclosure pertains having the benefit of the teachings presented in the foregoing descriptions and the associated drawings. Although specific terms may be employed herein, they are used in a generic and descriptive sense only and not for purposes of limitation. Accordingly, the present disclosure is not limited to the specific embodiments illustrated herein but is limited only by the following claims.

1. A field deployable modular decontamination station comprising:
a frame including side panels and a top panel, the side panels and the top panel are configured to be attached together to create the frame, the side panels and the top panel are configured to be folded or disassembled for transportation or shipping; and
a plurality of sprayers attached on an inside of the frame, the plurality of sprayers are configured to decontaminate a person or an object on the inside of the frame.

2. The field deployable modular decontamination station of claim 1, wherein each of the side panels and the top panel including a transparent window, the transparent windows are configured for observing the person or the object during a decontamination process on the inside of the frame.

3. The field deployable modular decontamination station of claim 1, wherein the frame further including a pan as a bottom of the frame, the pan is configured to hold and support the frame in an unfolded or assembled configuration and in a folded or disassembled configuration.

4. The field deployable modular decontamination station of claim 3, wherein the frame including the side panels, the top panel and the pan, is configured to be stored, transported, and deployed on a standard pallet; wherein the frame including the side panels, the top panel and the pan, is configured to be:
folded or disassembled by a single person; and
unfolded or assembled by the single person.

5. The field deployable modular decontamination station of claim 1, wherein the frame is configured to be modular, wherein the frame being modular is configured:
to allow the frame to be connected to an adjacent frame in a side-by-side or parallel configuration; or
to allow the frame to be connected to the adjacent frame in a front-to-back or series configuration.

6. The field deployable modular decontamination station of claim 5, wherein the frame being modular is configured to allow the frame to be:
connected to the adjacent frame in the side-by-side or parallel configuration; and
connected to the adjacent frame in the front-to-back or series configuration.

7. The field deployable modular decontamination station of claim 1, wherein each side panel including:
a lower side panel and an upper side panel configured to be stacked on top of each other, where the frame is configured as a tall frame configuration; or
a single high side panel, where the frame is configured as a short frame configuration.

8. The field deployable modular decontamination station of claim 7, wherein, when the tall frame configuration is provided and each side panel includes the lower side panel and the upper side panel, the lower side panel or the upper side panel of each of the side panels is configured to be removable to create the short frame configuration with the single high side panel on each of the side panels.

9. The field deployable modular decontamination station of claim 7, wherein:
the tall frame configuration is a human configuration configured and sized for decontaminating humans; or
the short frame configuration is a cart/gurney type configuration configured and sized for decontaminating shopping carts or hospital gurneys.

10. The field deployable modular decontamination station of claim 9, wherein the cart/gurney type configuration of the frame that is configured and sized for decontaminating the shopping carts or the hospital gurneys includes a removable top portion that is sized and configured for decontaminating the shopping carts or the hospital gurneys.

11. The field deployable modular decontamination station of claim 7, wherein the tall frame configuration is configured to be attached in a side-by-side or parallel configuration with an adjacent frame configured as the short frame configuration.

12. The field deployable modular decontamination station of claim 1, wherein the plurality of sprayers including:
at least one top sprayer attached to the top panel; and
at least one side sprayer attached to each of the side panels.

13. The field deployable modular decontamination station of claim 12, wherein:
the at least one top sprayer attached to the top panel including:
a front elongated top spray bar attached along a front top member on an inside of the top panel; and
a back elongated top spray bar attached along a back top member on the inside of the top panel;
the at least one side sprayer attached to each of the side panels including:
a front elongated side spray bar attached along a front side member on an inside of each of the side panels; and
a back elongated side spray bar attached along a back side member on the inside of each of the side panels.

14. The field deployable modular decontamination station of claim 12, wherein the plurality of sprayers further including at least one bottom sprayer positioned beneath a grate on the top of a pan at a bottom of the frame.

15. The field deployable modular decontamination station of claim 1 further including a pump configured to spray a disinfectant/sanitizing agent out of the plurality of sprayers.

16. The field deployable modular decontamination station of claim 15, wherein the pump is attached to the frame at a bottom corner of one of the side panels.

17. The field deployable modular decontamination station of claim 1 further including:
a card reader configured to scan or read a card of the person or the object;
a thermal scanning camera at a top configured to thermally scan the person or the object on the inside of the frame;
an enclosure on one or each side of the frame, each of the enclosures is configured to house a pump, tanks configured to hold a disinfectant/sanitizing agent, or combinations thereof;
a foot pressure pad at a bottom of the frame, the foot pressure pad is configured to sense presence of the person or the object on the inside of the frame;
ramps configured to provide a smooth surface from a ground to a pan at a bottom of the frame, the ramps are designed and configured to prevent the person from tripping on the pan while entering the frame and the ramps are designed and configured to let the object roll into the frame onto the pan from the ground;
or
combinations thereof.

18. The field deployable modular decontamination station of claim 17 including:
the card reader configured to scan or read the card of the person or the object;
the thermal scanning camera at the top configured to thermally scan the person or the object on the inside of the frame;
the enclosures on the one or the each side of the frame, the enclosures are configured to house the pump, the tanks configured to hold a disinfectant/sanitizing agent, or the combinations thereof;
the foot pressure pad at the bottom of the frame, the foot pressure pad is configured to sense the presence of the person or the object on the inside of the frame; and
the ramps configured to provide the smooth surface from the ground to the pan at the bottom of the frame, the ramps are designed and configured to prevent the person from tripping on the pan while entering the frame and the ramps are designed and configured to let the object roll into the frame onto the pan from the ground.

19. A field deployable modular decontamination station comprising:
a frame including side panels, a top panel and a pan, the side panels and the top panel are configured to be locked together to create the frame, the side panels and the top panel are configured to be folded or disassembled for transportation or shipping;
each of the side panels and the top panel including a transparent window, the transparent windows are configured for observing a person or an object during a decontamination process inside of the frame;
the pan is a bottom of the frame, the pan is configured to hold and support the frame in an unfolded or assembled configuration and in a folded or disassembled configuration;
a plurality of sprayers attached on an inside of the frame, the plurality of sprayers are configured to decontaminate the person or the object on the inside of the frame, the plurality of sprayer including:
at least one top sprayer attached to the top panel, the at least one top sprayer attached to the top panel including:
a front elongated top spray bar attached along a front top member on an inside of the top panel; and
a back elongated top spray bar attached along a back top member on the inside of the top panel;
at least one side sprayer attached to each of the side panels, the at least one side sprayer attached to each of the side panels including:
a front elongated side spray bar attached along a front side member on an inside of each of the side panels; and
a back elongated side spray bar attached along a back side member on the inside of each of the side panels;
at least one bottom sprayer positioned beneath a grate on the top of the pan at the bottom of the frame;
a pump configured to spray a disinfectant/sanitizing agent out of the plurality of sprayers, the pump is attached to the frame at a bottom corner of one of the side panels;
a card reader configured to scan or read a card of the person or the object;
a thermal scanning camera at a top configured to thermally scan the person or the object on the inside of the frame;
enclosures on one or each side of the frame, each of the enclosures are configured to house the pump and tanks configured to hold the disinfectant/sanitizing agent;
a foot pressure pad at a bottom of the frame, the foot pressure pad is configured to sense presence of the person or the object on the inside of the frame; and
ramps configured to provide a smooth surface from a ground to a pan at a bottom of the frame, the ramps are designed and configured to prevent the person from tripping on the pan while entering the frame and the ramps are designed and configured to let the object roll into the frame onto the pan from the ground;
wherein the frame including the side panels, the top panel and the pan, is configured to be stored, transported, and deployed on a standard pallet; wherein the frame including the side panels, the top panel and the pan, is configured to be:
folded or disassembled by a single person; and
unfolded or assembled by the single person;
wherein the frame is configured to be modular, wherein the frame being modular is configured:
to allow the frame to be connected to an adjacent frame in a side-by-side or parallel configuration; and
to allow the frame to be connected to the adjacent frame in a front-to-back or series configuration;
wherein, each side panel including:
a lower side panel and an upper side panel configured to be stacked on top of each other, where the frame is configured as a tall frame configuration, the tall frame configuration is a human configuration configured and sized for decontaminating humans; or
a single high side panel, where the frame is configured as a short frame configuration, the short frame configuration is a cart/gurney type configuration configured and sized for decontaminating shopping carts or hospital gurneys, the cart/gurney type configuration includes a removable top portion that is sized and configured for decontaminating the shopping carts or the hospital gurneys;
wherein, when the tall frame configuration is provided and each side panel includes the lower side panel and the upper side panel, the lower side panel or the upper side panel is configured to be removable to create the short frame configuration with the single high side panel; and
wherein the tall frame configuration is configured to be attached in the side-by-side or parallel configuration with the adjacent frame configured as the short frame configuration.

20. A method of decontaminating a person or an object comprising:
providing a field deployable modular decontamination station comprising:
a frame with a plurality of sprayers configured to decontaminate the person or the object on the inside of the frame;
the frame includes a plurality of panels configured to be folded or disassembled for transportation or shipping, the plurality of panels including side panels and a top panel configured to be locked together;
transporting the disassembled field deployable modular decontamination station to a desired location;
assembling the field deployable modular decontamination station at the desired location;
positioning the person or the object on the inside the frame; and
spraying the person or the object on the inside of the fame with the plurality of sprayers.
